(19) United States
(12) Patent Application Publication
(10) Pub. No.: US 2014/0347668 A1
(43) Pub. Date: Nov. 27, 2014

(54) PHANTOM AND METHOD FOR MANUFACTURING THE SAME, AND ACCURACY CONTROL METHOD

(71) Applicant: CANON KABUSHIKI KAISHA, Tokyo (JP)

(72) Inventor: Takahiro Suita, Kyoto-shi (JP)

(73) Assignee: CANON KABUSHIKI KAISHA, Tokyo (JP)

(21) Appl. No.: 14/282,984

(22) Filed: May 20, 2014

(30) Foreign Application Priority Data
May 23, 2013 (JP) 2013-108700

(51) Int. Cl. A61B 6/00; A61B 8/00

(52) U.S. Cl. CPC A61B 6/583; A61B 8/587
USPC 356/432; 359/599; 264/279.1

(57) ABSTRACT

A phantom has a base material, light absorbers/scatterers in the base material, and a film member on the surface of the base material. The base material has light propagation characteristics and acoustic propagation characteristics similar to those of a human tissue. The film member covers at least a portion of the base material. The phantom is for use with photoacoustic-wave diagnostic apparatus.

### BACKGROUND OF THE INVENTION

1. Field of the Invention

The present invention relates to a phantom for the control of the positional accuracy of photoacoustic-wave diagnostic apparatus and a method for manufacturing the phantom and also to an accuracy control method in which this phantom is used.

2. Description of the Related Art

Photoacoustic-wave diagnostic apparatus is an apparatus that displays an image based on a detection signal that corresponds to acoustic waves (typically, ultrasonic waves) generated while the biological body of interest thermally expands in response to irradiation with light. This type of diagnostic apparatus is used to examine selected substances in the site of interest, e.g., glucose, hemoglobin, and other substances in blood.

Medical diagnostic apparatus is used with a human tissue model, which is called a phantom, for the purposes of accuracy control and the training of technicians. The material that makes up a phantom should have characteristics similar to those of the human tissue and be able to be stored for long periods of time.

A phantom used with photoacoustic-wave diagnostic apparatus should be made of a material that has light propagation characteristics and acoustic propagation characteristics similar to those of a human tissue. For example, Japanese Patent Laid-Open No. 2011-209691 discloses a phantom for photoacoustic-wave diagnostic apparatus, and the base material of this phantom is a polyol that contains titanium oxide and carbon black dispersed therein.

The phantom for photoacoustic-wave diagnostic apparatus disclosed in Japanese Patent Laid-Open No. 2011-209691 is used in actual measurement with the tissue-equivalent material, mainly composed of the polyol, directly on the apparatus. Prior to this, the surface of the phantom is coated with water or ultrasonographic gel. This coating (water or gel) prevents the formation of air bubbles that could form in the gap between the phantom and the section of the apparatus where the phantom is placed. Repeated use of the phantom, however, leads to bacterial growth and other problems associated with the effects of moisture and air on the surface of the phantom, disadvantageously resulting in the degradation of the phantom.

In photoacoustic-wave mammography, in which the photoacoustic-wave technology is used to diagnose breast cancer, the site of interest, i.e., a breast, is compressed during immobilization. This means that a phantom used to control the accuracy of photoacoustic-wave mammography for the diagnosis of breast cancer also need to be compressed during immobilization in the apparatus. Repeated use of the phantom therefore causes cracks and other defects to occur in the phantom, disadvantageously affecting the control of the accuracy of the apparatus.

To solve these problems, an aspect of the invention is intended to provide a phantom that can be used with photoacoustic-wave diagnostic apparatus for long periods of time without degradation.

### SUMMARY OF THE INVENTION

A phantom according to an aspect of the invention has: a base material having light propagation characteristics and acoustic propagation characteristics similar to those of a human tissue; a light absorber/scatterer in the base material; and a film member on the surface of the base material, the film member covering at least a portion of the base material, the phantom being for use with photoacoustic-wave diagnostic apparatus.

Further features of the present invention will become apparent from the following description of exemplary embodiments with reference to the attached drawings.

### BRIEF DESCRIPTION OF THE DRAWINGS

FIG. 1 is a schematic diagram that illustrates a detailed structure of photoacoustic diagnostic apparatus.

FIG. 2 is a schematic diagram that illustrates a first embodiment of a phantom according to an aspect of the invention.

FIG. 3 is a schematic diagram that illustrates a second embodiment of a phantom according to an aspect of the invention.

FIG. 4 is a schematic diagram that illustrates a detailed structure of breast-shaped photoacoustic diagnostic apparatus.

FIG. 5 is a schematic diagram that illustrates a third embodiment of a phantom according to an aspect of the invention.

### DESCRIPTION OF THE EMBODIMENTS

The following describes some embodiments of the invention with reference to the drawings. Each of the embodiments described below is just one of a number of embodiments of the invention, and no aspect of the invention is limited to these embodiments.

In a certain embodiment of the invention, a biological body, e.g., a human body, is not limited to a living body. Naturally, resected lesion sites such as a cancer or a tumor are also included.

### Example 1

### Phantom

A phantom according to an embodiment of the invention is a phantom for use with photoacoustic-wave diagnostic apparatus. A phantom according to an embodiment of the invention has a base material whose light propagation characteristics and acoustic propagation characteristics are similar to those of a human tissue, a light absorber/scatterer in the base material, and a film member on the surface of the base material. At least a portion of the base material is covered with this film member.

In an embodiment of the invention, it is possible that both of the base material and the light absorber/scatterer that make up the phantom are made of a polyol.

In an embodiment of the invention, examples of polyols that can be used to make the base material and the light absorber/scatterer include polyether polyols, polyester polyols, and polycarbonate polyols. In particular, polyether polyols exhibit a good correlation concerning the acoustic propagation characteristics of a human tissue. Polyether polyols that have an ethylene oxide unit and a propylene oxide unit can also be used. The base material and the light absorber/scatterer can be made of a polyether polyol in which the molar ratio between the ethylene oxide unit and the propylene oxide unit is from 30:70 to 70:30. This is a range that can be applicable when the correlation concerning the acoustic propagation characteristics of a human tissue and the stability of the polymer are considered. The number-average molecular weight of a polyol used to make the base material and the light absorber/scatterer can be from 5000 to 8000.

A polyol used to make a phantom according to an embodiment of the invention is usually a liquid. Adding a curing agent as necessary to make the polyol turn into a solid is a possible way to form a solid phantom. Such a curing agent used to form a solid phantom can be of any kind. From the viewpoint of ensuring acoustic propagation characteristics similar to those of a human tissue, such a curing agent can be an isocyanate compound. Specific examples of isocyanate compounds include hexamethylene diisocyanate (HDI), diphenylmethane diisocyanate (MDI), tolylene diisocyanate (TDI), isophorone diisocyanate (IPDI), and xylylene diisocyanate (XDI). Although the following description of this embodiment discusses a case where a polyol is cured into a polymeric material with the use of HDI, isocyanate compounds other than HDI may be used in other embodiments of the invention.

A phantom according to an embodiment of the invention may have filler dispersed in the base material.

Making the light propagation characteristics of the base material and the light absorber/scatterer that make up the phantom similar to those of a human tissue requires adjusting the effective scattering coefficient and the absorption coefficient. A possible way to do this adjustment is to disperse filler in the base material.

Such filler dispersed in the base material may be one that has light-scattering properties. Examples of light-scattering fillers include inorganic oxides such as titanium oxide.

When one tries to disperse filler in the base material, (some of) the filler may aggregate as a precipitate or in other forms without being dispersed in the base material. An example of a way to prevent this aggregation of filler is to modify the surface of the filler. For example, in a case where titanium oxide is used as filler, the titanium oxide precipitates in the polyol and is difficult to disperse uniformly if no surface modification has been made. If the surface thereof has been modified, however, the titanium oxide successfully disperses in the base material with no such aggregation or precipitation occurring.

This surface modification technique, which can be applied to all materials that are commonly used as light-scattering fillers, is particularly effective when the filler is a metal oxide, such as titanium oxide. An example of a way to modify the surface of filler is surface treatment with aluminum oxide and hexamethyldisilazane.

When a light-scattering filler is used, the average particle diameter of the filler can be from 0.1 μm to 0.3 μm for favorable diffusion of light.

Examples of fillers that can be dispersed in the base material other than metal oxides such as titanium oxide include pigments.

Pigment can be used as a light-absorbing filler. Examples of pigments that can be used as filler include black pigments such as carbon black, cyan pigments such as copper phthalocyanine, magenta pigments such as monoazo lake pigments and monoazo pigments, and yellow pigments such as diarylide yellow. However, trying to disperse pigment directly in the base material may cause at least some of the pigment aggregate as a precipitate or in other forms without being dispersed in the base material because of the compatibility of the pigment with the material that makes up the base material.

In such a case, the filler can be a pigment that forms a covalent bond with the polyol so that the pigment, i.e., the filler, can be dispersed faster. This is particularly effective when the filler is a pigment based on a carbon-containing compound, such as carbon black. Naturally, pigments based on a carbon-containing compound include pigments based on an organic compound.

An example of a way to join a polyol and pigment with a covalent bond is to form an ether bond (—O—) or an ester bond (—O—(C═O)—) through chemical reaction from a terminal substituent of the polyol, i.e., a hydroxyl group (—OH).

When pigment is used as filler, furthermore, the pigment can be used in the form of a dispersion of a complex of the polyol and the pigment connected with a covalent bond.

In an embodiment of the invention, the amount of filler dispersed can be from 0.1% to 0.5% by weight relative to the polyol used in the phantom.

In an embodiment of the invention, the base material and the light absorber/scatterer that make up the phantom contain a polyol and can also contain a curing agent and filler. Although the base material and the light absorber/scatterer are thus equivalent in terms of the basic constituents, the proportions of the constituents in each component may vary.

In an embodiment of the invention, the film member, with which at least a portion of the base material is covered, can be made of a material that is less water-absorbent than the base material and excellent in terms of tear strength and other mechanical characteristics. Furthermore, the film member can be made of a material that has little influence on the light propagation characteristics and the acoustic propagation characteristics of the base material during measurement. In an embodiment of the invention, the film member can be made from polymer film. In particular, a polymer film made of polymethylpentene has high transparency and causes only limited acoustic attenuation. Other kinds of polymer films can also be used.

In an embodiment of the invention, the thickness of the film member can be from 0.1 mm to 3 mm so that measurements will not be affected. It is also possible to use a multilayer film or disperse filler in the film for purposes such as making the film member more similar to the skin structure. Examples of ways to cover the base material with the film member include pouring the base material into an enclosure, bonding the film member to the surface of the base material, and processing the surface of the base material using an additional material to form the film member. When a way in which the base material is poured into an enclosure is used, examples of enclosures that can be used include those made from the film member alone and those having the film member and a support. Enclosures that have the film member and a support would have sufficient strength.

### (2) Method for Manufacturing a Phantom

The following describes a method for preparing (manufacturing) a phantom according to this embodiment. A phantom according to this embodiment can be prepared by the method described below, for example.

Filler was dispersed in a beaker that contained a polyol. The resulting dispersion was stirred and then degassed under vacuum. The polyol was a polyether polyol copolymer in which the molar ratio between ethylene oxide and propylene oxide was 1:1 (number-average molecular weight: 7000). The following is a list of filler materials used:

Titanium oxide (surface-treated with aluminum oxide and hexamethyldisilazane; average particle diameter, 0.21 μm), 0.24% by weight relative to the polyol;
A black pigment, 0.0002% by weight relative to the polyol.

Then a curing agent (HDI), 3.0% by weight relative to the polyol, was added. The resulting mixture was stirred and then degassed under vacuum.

Then the polyol, to which the curing agent has been added, is poured into a selected mold and cured by heating. More specifically, a polymer film (0.5-mm thick polymethylpentene film) was placed in an aluminum die, the polyol that contained the filler and the curing agent was poured, and then the polyol was heated at 90° C. for 1 hour. This makes the polyol cure and form the base material.

A light absorber/scatterer to be detected is placed in the phantom by placing the light absorber/scatterer in the aforementioned die, pouring the polyol into the die, and then curing the polyol. The light absorber/scatterer may have a hardness higher than that of the base material so that the effects of the condensation of the polyol and other events that occur while the polyol is cured will be reduced. In this example, a light absorber/scatterer harder than the base material was prepared by dispersing a curing agent, 3.4% by weight relative to the polyol, in a polyol that had the same composition as that in the tissue-equivalent material (base material). Then during the formation of the base material, the light absorber/scatterer was placed in a predetermined position in the die before the polyol was poured into the die.

### (3) Evaluation of the Characteristics of the Phantom (Calculations of the Light Propagation Characteristics)

The following describes the method by which the light propagation characteristics of the phantom prepared in this example were evaluated. A cell for the measurement of light propagation characteristics was prepared by pouring the filler-dispersed liquid polyol to which the curing agent had been added into a 50 mm×50 mm quartz cell that had an optical path length of 5 mm and curing the polyol by heating at 90° C. for 1 hour. Then the permeability and the reflectivity of this cell was determined with JASCO V-670 spectrophotometer. The refractive index of the cured polymer was determined through analysis of a separately prepared sample of the cured polymer (size: 10 mm×10 mm×50 mm) with a refractometer available from Shimadzu Corporation (KPR-2000). The obtained results were simulated by the Monte Carlo method and the parameters were optimized to minimize the differences between the measurements and the calculations. In this way, the effective scattering coefficient and the absorption coefficient at each wavelength were calculated.

The following describes the method by which acoustic propagation characteristics were evaluated in an embodiment of the invention. The ultrasonic transducer (a transmitter unit) used as a probe in the evaluation of acoustic propagation characteristics was Olympus NDT V303 (center frequency: 1 MHz). The hydrophone (a receiver unit) was Precision Acoustics PAL-1384 needle hydrophone. The transducer and the hydrophone were fastened in a water tank by using a jig, with the center of their sound axis aligned. The distance between the transducer and the hydrophone was 40 mm.

The acoustic propagation characteristics of the cured polyol were evaluated by the following method. A sheet with a size of 100 mm×100 mm and a thickness of 5 mm or 10 mm prepared from the cured polyol was fastened between the transducer and the hydrophone in the above experimental system with the use of a jig in such a manner that the angle of incidence of the ultrasonic signal on the sheet should be 0°. One cycle of a sine wave (transmission voltage: 100 V) was then transmitted from the transducer by using a function generator (Tectronix AFG3022). The value of the voltage received by the hydrophone was determined with an oscilloscope (Tectronix TDS 3012C) for each of the sheets. The speed of sound was determined with the oscilloscope. More specifically, the speed of sound was determined from the difference between the time of travel of the received wave measured with the cured polyol sheet in the measurement system and that with no cured polyol sheet. The acoustic attenuation was determined by the following equation:

$$Acoustic attenuation per cm per MHz ( dB \text{/} cm \text{/} MHz ) = 20 \times log [ \frac{Sound pressure received with a 10 \text{-} mm thick sheet placed}{Sound pressure received with a 5 \text{-} mm thick sheet placed} ] \times \frac{10 ( mm )}{5 ( mm )}$$

### (4) Examples in which the Phantom was Used with Photoacoustic-Wave Diagnostic Apparatus

The following describes, with reference to drawings, some examples in which a phantom according to an embodiment of the invention was used to control the accuracy of photoacoustic-wave diagnostic apparatus.

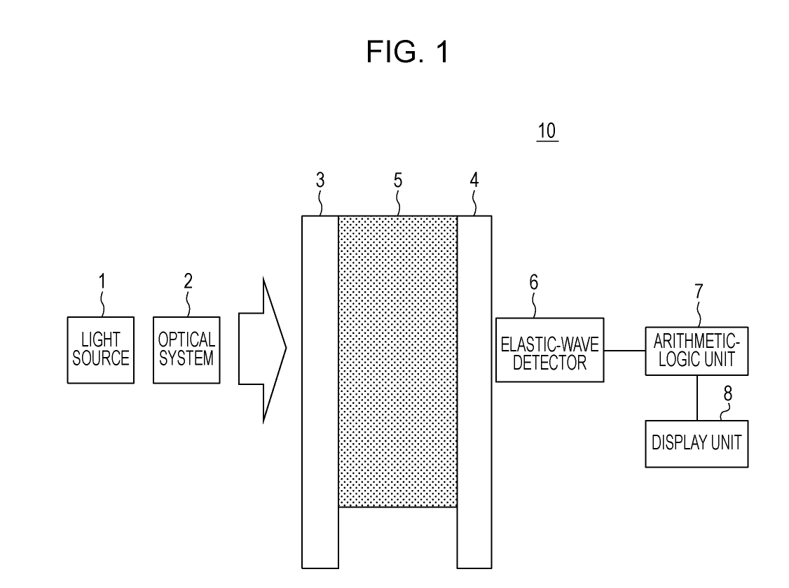

FIG. 1 illustrates an example of the structure of photoacoustic-wave diagnostic apparatus. Photoacoustic-wave diagnostic apparatus 10 in FIG. 1 has a light source 1, an optical system 2, a first holding plate 3, a second holding plate 4, a phantom 5, an elastic-wave detector 6, an arithmetic-logic unit 7, and a display unit 8. In the photoacoustic-wave diagnostic apparatus 10 in FIG. 1, the phantom 5 is sandwiched between the first holding plate 3 and the second holding plate 4. Since the phantom 5 is located between the first holding plate 3 and the second holding plate 4, the first holding plate 3 and the second holding plate 4 are in a certain distance from each other.

The following describes the individual elements of the photoacoustic-wave diagnostic apparatus 10 in FIG. 1 in detail.

The light source 1 is used to irradiate the object (e.g., the phantom 5) with nanosecond light pulses of particular wavelengths. The light emitted from the light source 1 is selected in such a manner that the wavelengths of the light should match the absorption spectrum of water, fat, hemoglobin, or any other constituent of a biological tissue. For example, when the object is hemoglobin in a blood vessel, the 600 nm to 1100 nm range is suitable because the absorption spectrum of blood oxyhemoglobin and deoxyhemoglobin has a distinctive shape over this range. Specific examples of the light source 1 include semiconductor lasers with which multiple wavelengths can be generated and wavelength-tunable lasers. In this example, the light source 1 was a titanium-sapphire (Ti—S) laser.

The optical system 2 is provided to guide the light emitted from the light source 1 to the object (e.g., the phantom 5). The optical system 2 is made up of, for example, optical fibers and a lens. The light emitted from the light source 1 is magnified through the optical system 2 on the whole area of the interface between the first holding plate 3 and the object and guided to the surface of the object through the first holding plate 3. In this example, the optical system 2 was a lens.

The first holding plate 3 and the second holding plate 4 may be able to allow the light emitted from the light source 1 to efficiently pass through it (high permeability) and able to allow acoustic waves to pass through it with as little loss as possible (low attenuation). Examples of materials that can be used as the first holding plate 3 and the second holding plate 4 include glass, polymethylpentene, polycarbonate, and acrylic resin. In this example, the first holding plate 3 and the second holding plate 4 were made of polymethylpentene.

The phantom 5 is held (in compression) between the first holding plate 3 and the second holding plate 4. Applying water or gel to the surface of the phantom 5 will prevent air bubbles from forming in the gaps between the phantom 5 and the holding plates 3 and 4.

The arithmetic-logic unit 7 has a memory, in which the optical coefficient, a value relating to the light absorber/scatterer placed in the phantom 5, obtained by the parametric optimization mentioned in the description of the method for calculating light propagation characteristics is stored as a true value. In this example, the arithmetic-logic unit 7 had a calculation section, which compared measurements with the true value, a correction section, which corrected measurement errors, and an error determination section, which determined a measurement to be an error if the measurement was significantly different from the true value.

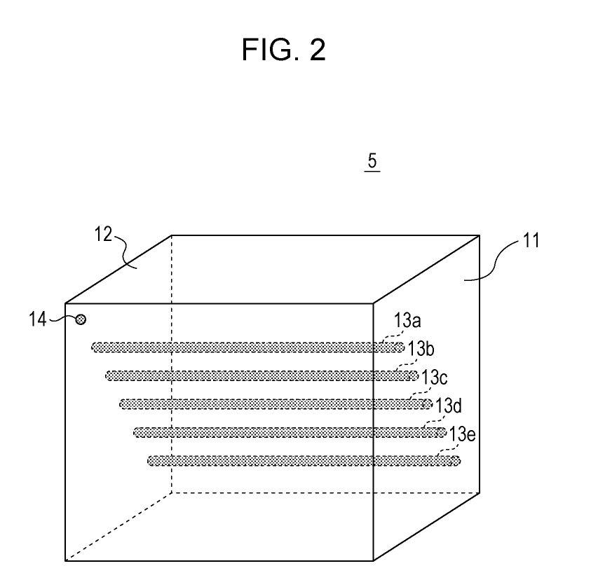

FIG. 2 is a schematic diagram that illustrates a first embodiment of a phantom according to an aspect of the invention. The phantom 5 in FIG. 2 is a diagram that schematically illustrates the phantom according to this example for photoacoustic-wave diagnostic apparatus. The phantom 5 in FIG. 2 has a base material 11, a film member 12, and light absorbers/scatterers (13a to 13e). The base material 11 is made of a medium whose light propagation characteristics and acoustic propagation characteristics are both similar to those of a human tissue. The film member 12 is a component with which the surface of the base material 11 is covered. In this example, the film member 12 was made from 0.5-mm thick polymethylpentene film. The light absorbers/scatterers (13a to 13e), all located in the base material 11, are for use as the object of the detection of model blood vessels. The phantom 5 also has an alignment mark 14 on the surface thereof, and this mark is used to position the phantom 5 in the apparatus. In an embodiment of the invention, the alignment mark 14 may be located on the film member 12.

In this example, the size of the phantom 5 was 120 mm×120 mm×50 mm. The size of the multiple light absorbers/scatterers (13a to 13e) in the phantom 5 was a cylinder with a diameter of 2 mm and a length of 120 mm. The light absorbers/scatterers (13a to 13e) were positioned in the phantom 5 in such a manner that the light absorbers/scatterers (13a to 13e) could be detected at depths of 5 mm, 15 mm, 25 mm, 35 mm, and 45 mm. A 2-mm diameter sphere made of the same material as the light absorbers/scatterers was placed at a predetermined point on the outer surface of the film member 12 to provide an alignment mark 14.

The absorption coefficient and the effective scattering coefficient of the base material 11 and the light absorbers/scatterers 13 of the phantom 5 in FIG. 2 were measured at measurement wavelengths of 756 nm and 797 nm. The results are summarized in the Table.

TABLE

| | | Light |
|---|---|---|
| | Base | absorbers/ |
| | material | scatterers |
| Absorption coefficient (@756 nm) [mm⁻¹] | 0.0039 | 0.024 |
| Effective scattering coefficient (@756 nm) [mm⁻¹] | 1.10 | 1.10 |
| Absorption coefficient (@797 nm) [mm⁻¹] | 0.0028 | 0.021 |
| Effective scattering coefficient (@797 nm) [mm⁻¹] | 1.03 | 1.03 |

The evaluation of the acoustic propagation characteristics of the base material 11 of the phantom 5 in FIG. 2 revealed that the speed of sound was 1393.7 m/s and the acoustic attenuation was 0.38 dB/cm/MHz. The base material 11 of the phantom 5 in this example was therefore found to have light propagation characteristics and acoustic propagation characteristics almost identical to those of a human tissue.

Furthermore, the light absorbers/scatterers 13 in the phantom 5 were found to be suitable for the detection of hemoglobin because the absorption coefficient μₐ of red blood cells at a wavelength of 797 nm is approximately 0.02 mm⁻¹.

The following is a detailed description of a method for controlling the accuracy of photoacoustic-wave diagnostic apparatus and correcting the apparatus using the phantom prepared in this example. For example, if a measured absorption coefficient of the light absorber/scatterer 13a in the phantom 5 at 797 nm is 0.027, it is possible to calculate that the absorption coefficient error ratio for the light absorber/scatterer 13a is 0.027/0.021, i.e., 1.29. The absorption coefficient error ratio is also calculated for the other light absorbers/scatterers (13b to 13e) in the same way, and this allows the distribution of errors to be determined across the individual measurement points in the photoacoustic-wave diagnostic apparatus. An inaccuracy threshold is also provided. For example, a function is provided that determines the apparatus to lack accuracy if the error of the measurement from the true value is 50% or more. In this way, the accuracy of photoacoustic-wave diagnostic apparatus can be controlled with the use of the phantom prepared in this example and an accuracy control method.

The base material of the phantom prepared in this example has a coefficient of water absorption of 7.2% by weight (ambient temperature, 24 hours) and a tear strength of 1.7 MPa. Hence repeated use of the phantom prepared in this example leads to an increased detection error of the light absorbers/scatterers as a result of bacterial growth and cracks occurring on the surface of the base material in about one month. Long and continuous use of the base material therefore makes the base material no longer suitable for the control of the accuracy of photoacoustic-wave diagnostic apparatus. The phantom in this example has the base material thereof covered with a film member made of polymethylpentene. Polymethylpentene, i.e., the film member, had a coefficient of water absorption of 0.01% by weight or less (ambient temperature, 24 hours) and a tear strength of 30 MPa. A phantom according to an embodiment of the invention can therefore be used, for the purpose of controlling the accuracy of photoacoustic-wave diagnostic apparatus, in a stable manner for about one year.

### Example 2

A phantom was prepared in the same way as in Example 1 except that the film member 12 was used as an enclosure for the base material 11 in Example 1. The following describes the structure of the phantom according to this example (Example 2) for photoacoustic-wave diagnostic apparatus and the method by which this phantom was prepared.

The size and the constituents of the base material 11 and the light absorbers/scatterers (13a to 13e) of the phantom 5 prepared in this example are the same as those for the phantom in Example 1. A mold corresponding to the die was prepared with the use of a 3-mm thick polymethylpentene sheet, and then the light absorbers/scatterers (13a to 13e) were each placed in this mold in the same way as in Example 1. Then the polyol (containing a curing agent) from which the base material 11 would be made was poured into this mold and heated until the polyol cured. A holding plate (not illustrated) was then placed over the cured polyol. The holding plate was placed over the polyol with the interface between the phantom and the holding plate filled with a gel sheet made of polyurethane so that air bubbles should be prevented from forming. In this way, a phantom 5 was prepared that had a base material 11 having light and acoustic propagation characteristics equivalent to those of a human tissue in an enclosure made of polymethylpentene (the film member 12).

The obtained phantom was used to control the accuracy of photoacoustic-wave diagnostic apparatus and correct the apparatus in the same way as in Example 1.

As in Example 1, the phantom prepared in this example can be used to control the accuracy of photoacoustic-wave diagnostic apparatus. In addition to this, the use of a structure in which an enclosure made from the film member 12 covers the surface of the base material of the phantom prevents the degradation of the phantom due to water and compression. Furthermore, the light absorbers/scatterers (13a to 13e) placed in the phantom for photoacoustic-wave diagnostic apparatus in this example are hardly affected by compression while the phantom is positioned in photoacoustic-wave diagnostic apparatus, and this leads to improved positional accuracy in the detection of the light absorbers/scatterers.

### Example 3

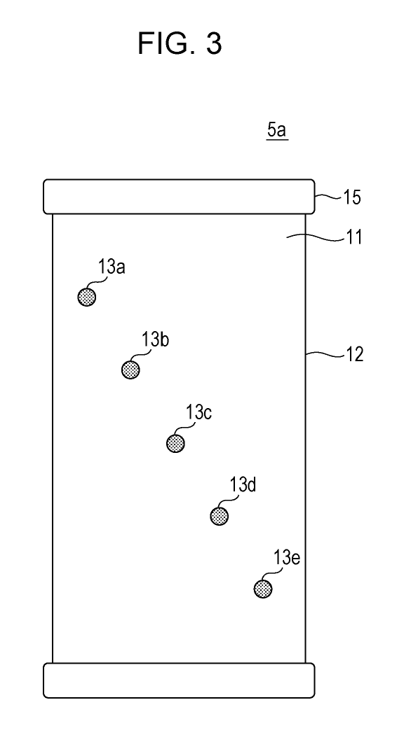

FIG. 3 is a schematic diagram that illustrates a second embodiment of a phantom according to an aspect of the invention. The phantom 5a in FIG. 3 illustrates the structure of the phantom according to this example (Example 3) for photoacoustic-wave diagnostic apparatus. The size and the constituents of the base material 11 and the light absorbers/scatterers (13a to 13e) of the phantom 5a in FIG. 3 were the same those for the phantom in Example 1. The phantom 5a in FIG. 3 also had a support 15, having a diameter of 10 mm and a length of 50 mm and made of aluminum, at each of the four corners. In the phantom 5a in FIG. 3, the light absorbers/scatterers (13a to 13e) in the phantom 5a were placed in the same manner as in Example 1 (FIG. 2). The phantom 5a in this example was, like the phantom in Example 2, was covered with a holding plate with the interface between the phantom and the holding plate filled with a gel sheet made of polyurethane so that air bubbles should be prevented from forming.

The obtained phantom was used to control the accuracy of photoacoustic-wave diagnostic apparatus and correct the apparatus in the same way as in Example 1.

The phantom prepared in this example, like those in Example 1 and Example 2, can be used to control the accuracy of photoacoustic-wave diagnostic apparatus. Furthermore, the light absorbers/scatterers (13a to 13e) used in the phantom in this example are hardly affected by compression while the phantom is positioned in photoacoustic-wave diagnostic apparatus, and this leads to improved positional accuracy in the detection of the light absorbers/scatterers.

### Example 4

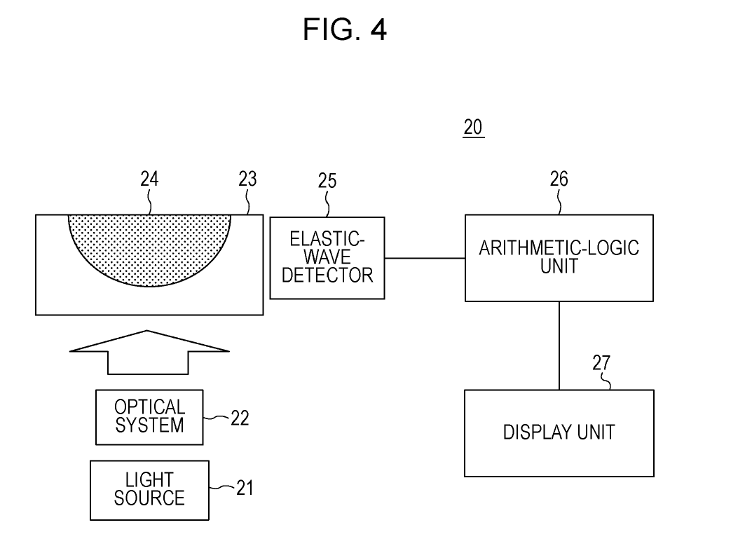

FIG. 4 is a schematic diagram that illustrates a detailed structure of a breast-shaped photoacoustic diagnostic apparatus.

The breast-shaped photoacoustic-wave diagnostic apparatus 20 in FIG. 4 has a light source 21, an optical system 22, an object-holding section 23, a phantom 24, an elastic-wave detector 25, an arithmetic-logic unit 26, and a display unit 27. The components of the breast-shaped photoacoustic-wave diagnostic apparatus 20 in FIG. 4 excluding the object-holding section 23 correspond to the components of the photoacoustic-wave diagnostic apparatus 10 in FIG. 1 (excluding the first holding plate 3 and the second holding plate 4). The object-holding section 23 of the breast-shaped photoacoustic-wave diagnostic apparatus 20 in FIG. 4 has a hemispherical depression that fits over the shape of the phantom 24 placed thereon (a hemisphere having a radius of 100 mm).

An example of the shape of the phantom 24 is a hemisphere having a radius of 100 mm. However, the shape of a phantom is not limited to this in any aspect of the invention.

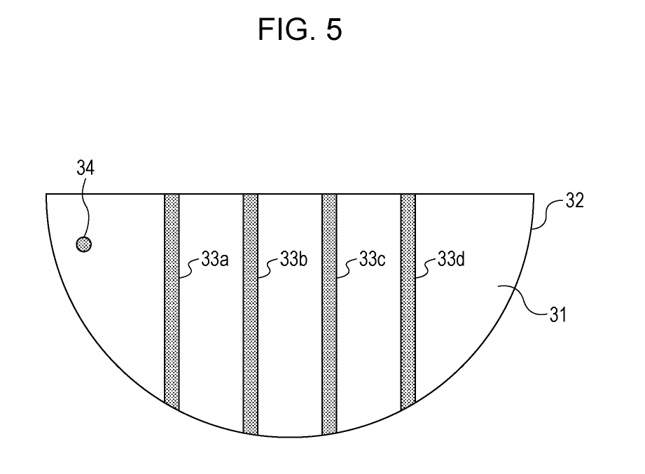

FIG. 5 is a schematic diagram that illustrates a third embodiment of a phantom according to an aspect of the invention. The phantom 24 in FIG. 5 is a phantom that can be used with the breast-shaped photoacoustic-wave diagnostic apparatus 20 in FIG. 4.

The phantom 24 in FIG. 5 has a base material 31, a film member 32, and light absorbers/scatterers (33a to 33d). The base material 31 of the phantom 24 in FIG. 5 is made of a medium that has light propagation characteristics and acoustic propagation characteristics similar to those of a human tissue. The film member 32 is a component with which the surface of the base material 31 is covered and is made from 0.5-mm thick polymethylpentene film. The film member 32 of the phantom 24 has an alignment mark 34 at the predetermined point indicated in FIG. 5, and this mark is used to position the phantom 24 in the object-holding section 23 of the breast-shaped photoacoustic diagnostic apparatus 20 in FIG. 4. The light absorbers/scatterers (33a to 33d), located at the predetermined points in FIG. 5 in the base material 31, are for use as the object of the detection of model blood vessels.

In this example, the size of the phantom 24 was a hemisphere with a radius of 100 mm. The size of the light absorbers/scatterers (33a to 33d) in the phantom 24 was a cylinder with a diameter of 2 mm and a length of 100 mm. The phantom 24 was positioned in the object-holding section 23 of the breast-shaped photoacoustic diagnostic apparatus 20 in FIG. 4 in such a manner that the light absorbers/scatterers (33a to 33d) could be detected at regular spatial intervals in the middle as illustrated in FIG. 5. A 2-mm diameter sphere was placed on the phantom 24 in FIG. 5 to provide an alignment mark 34. The alignment mark 34 was made of the same material as the light absorbers/scatterers (33a to 33d). The constituents of the base material 31 and the film member 32 were the same as those for the phantom prepared in Example 1. In this way, the breast-shaped phantom illustrated in FIG. 5 was obtained.

The obtained phantom was used to control the accuracy of photoacoustic-wave diagnostic apparatus and correct the apparatus in the same way as in Example 1.

The phantom 24 prepared in this example, like the phantoms in the other examples (Example 1 to Example 3), can be used to control the accuracy of photoacoustic-wave diagnostic apparatus. In addition to this, the structure of the phantom prepared in this example, in which an enclosure made from the film member 32 covers the surface of the base material 31, prevents the degradation of the phantom 24 due to water and compression. Furthermore, the light absorbers/scatterers (33a to 33d) of the phantom in this example are hardly affected by compression while the phantom is positioned in photoacoustic-wave diagnostic apparatus, and this leads to improved positional accuracy in the detection of the light absorbers/scatterers.

### Example 5

A phantom was prepared in the same way as in Example 4 except that the film member 32 was used as an enclosure for the base material 31 in Example 4. The following describes the structure of the phantom for photoacoustic-wave diagnostic apparatus according to this example (Example 5) and the method by which this phantom was prepared.

The size and the constituents of the base material and the light absorbers/scatterers of the phantom prepared in this example are the same as those for the phantom 24 in Example 4. A mold corresponding to the die was prepared with the use of a 3-mm thick polymethylpentene sheet, and then the light absorbers/scatterers were each placed in this mold in the same way as in Example 4. Then the polyol (containing a curing agent) from which the base material would be made was poured into this mold and heated until the polyol cured. A holding plate (not illustrated) was then placed over the cured polyol. The holding plate was placed over the polyol with the interface between the phantom and the holding plate filled with a gel sheet made of polyurethane so that air bubbles should be prevented from forming. In this way, a phantom was prepared that had a base material having light and acoustic propagation characteristics equivalent to those of a human tissue in an enclosure made of polymethylpentene (the film member).

The phantom prepared in this example, like those in the other examples (Example 1 to Example 4), can be used to control the accuracy of photoacoustic-wave diagnostic apparatus. In addition to this, the structure of the phantom prepared in this example, in which an enclosure made from the film member covers the surface of the base material, prevents the degradation of the phantom due to water and compression. Furthermore, the light absorbers/scatterers of the phantom in this example are hardly affected by compression while the phantom is positioned in photoacoustic-wave diagnostic apparatus, and this leads to improved positional accuracy in the detection of the light absorbers/scatterers.

An aspect of the invention provides a phantom that can be used with photoacoustic-wave diagnostic apparatus for long periods of time without degradation. This means that a phantom according to an aspect of the invention has a base material that is unlikely to be degraded and improves accuracy in the control of apparatus based on a signal generated by a light absorber/scatterer in the phantom.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-108700 filed May 23, 2013, which is hereby incorporated by reference herein in its entirety.

What is claimed is:

1. A phantom comprising:
a base material having light propagation characteristics and acoustic propagation characteristics similar to light propagation characteristics and acoustic propagation characteristics of a human tissue;
a light absorber/scatterer in the base material; and
a film member on a surface of the base material, the film member covering at least a portion of the base material,
the phantom being for use with photoacoustic-wave diagnostic apparatus.

2. The phantom according to claim 1, wherein the base material and the light absorber/scatterer are both made of a polyol.

3. The phantom according to claim 2, wherein:
the polyol is a polyether polyol that has an ethylene oxide unit and a propylene oxide unit; and
a molar ratio between the ethylene oxide unit and the propylene oxide unit is from 30:70 to 70:30.

4. The phantom according to claim 2, wherein a number-average molecular weight of the polyol is from 5000 to 8000.

5. The phantom according to claim 1, further comprising filler dispersed in the base material.

6. The phantom according to claim 5, wherein the filler is surface-modified titanium oxide.

7. The phantom according to claim 5, wherein the filler is a pigment that forms a covalent bond with the polyol.

8. The phantom according to claim 5, wherein a content of the dispersed filler is from 0.1% to 0.5% by weight relative to the polyol.

9. The phantom according to claim 1, further comprising an alignment mark on the film member.

10. The phantom according to claim 1, wherein the film member is made from polymer film.

11. The phantom according to claim 1, wherein the base material is contained in an enclosure.

12. The phantom according to claim 11, wherein the enclosure has the film member and a support.

13. A method for manufacturing the phantom according to claim 1, the method comprising:
placing the light absorber/scatterer in the base material; and
covering the base material with the film member,
the base material having light propagation characteristics and acoustic propagation characteristics similar to light propagation characteristics and acoustic propagation characteristics of a human tissue,
the phantom being for use with photoacoustic-wave diagnostic apparatus.

14. The method for manufacturing a phantom according to claim 13, further comprising
pouring a polyol into an enclosure for the base material,
the enclosure made from the film member and containing the light absorber/scatterer.

15. A method for controlling accuracy of photoacoustic-wave diagnostic apparatus with use of the phantom according to claim 1, the method comprising
correcting a measurement or determining a measurement to be an error by comparing the measurement with an absorption coefficient of the light absorber/scatterer in the phantom.
